(19) United States
(12) Patent Application Publication
(10) Pub. No.: US 2024/0368714 A1
(43) Pub. Date: Nov. 7, 2024

(54) COMPOSITION, KIT, METHOD FOR DETECTING HIV-1 AND USE THEREOF

(71) Applicant: Sansure Biotech Inc., Changsha (CN)

(72) Inventors: Lizhong DAI, Changsha (CN); Feng DING, Changsha (CN); Xiaoming LIU, Changsha (CN); Xiaomei REN, Changsha (CN); Jia LIU, Changsha (CN); Zhongping DENG, Changsha (CN)

(21) Appl. No.: 18/777,153

(22) Filed: Jul. 18, 2024

Related U.S. Application Data
(63) Continuation-in-part of application No. PCT/CN2022/135446, filed on Nov. 30, 2022.

(30) Foreign Application Priority Data
Jan. 27, 2022 (CN) 202210102254.9

(51) Int. Cl. C12Q 1/70

(52) U.S. Cl. CPC C12Q 1/703

(57) ABSTRACT

The present application belongs to the field of molecular biology detection, in particular, relates to detection of HIV-1. The present application further provides a kit comprising the composition of the present application, use of the composition and a method for detecting HIV-1. Using the composition of the present application, HIV-1 can be detected with a sensitivity of 22 IU/ml. Meanwhile, in the composition of the present application, two sets of primers and probes for two different regions and an additional forward primer are combined to prepare a reaction system and the HIV-1 RNA are detected using the reaction system to identify the positive reactions, effectively reducing the probability of false negative, especially, of group O viruses. There is no need for a fully enclosed diagnostic system, which effectively reduces costs.

### CROSS-REFERENCE

The application is a continuation in part of International Patent Application No. PCT/CN2022/135446, filed Nov. 30, 2022, which claims the benefit of China Patent Application No. 202210102254.9, filed Jan. 27, 2022, all of which are incorporated herein by reference in their entirety.

### SEQUENCE LISTING

The present application contains a Sequence Listing which has been submitted electronically in XML format and is hereby incorporated by reference in its entirety. The XML copy, created on Jul. 7, 2024, is named 68587-701.501_SL.xml and is 31,643 bytes in size.

### TECHNICAL FIELD

The present application belongs to the field of molecular biological detection. Particularly, the present application relates to the detection of HIV-1.

### BACKGROUND

Acquired immune deficiency syndrome (AIDS) is caused by the human immunodeficiency virus (HIV). HIV is an enveloped virus that encodes two envelope (Env) glycoproteins (the surface (SU) and transmembrane (TM) subunits), four major Gag proteins (matrix (MA), capsid (CA), nucleocapsid (Nc), and p6), and the pol-encoded enzymes (protease (PR), reverse transcriptase (RT), and integrase (IN)). Like other replication-competent retroviruses, to initiate the process of virus assembly, HIV uses the above polyprotein precursors encoded by the gag, and pol genes. AIDS is one of the important public health problems affecting public health. HIV is a highly mutated virus with each gene having different degrees of mutation. The main reasons for HIV mutation comprise random mutagenesis caused by the reverse transcriptase having no proofreading ability; virus replication in vivo at high frequency; immune selective pressure of the host; genetic recombination between viral DNA and host DNA; and drug selective pressure. Non-standard ART and poor patient compliance are main cause for drug resistance variation. HIV type 1 (HIV-1) is a subtype of HIV and can be stratified into major group (group M), outlier group (group O), non-M/non-O group (group N), and “pending the identification of further human cases” group (group P). HIV-1 group O have diverged from the HIV group M, with up to about 30% sequence differences in the pol gene, respectively.

Laboratory tests for HIV/AIDS patients mainly comprise HIV antibody tests, HIV nucleic acid qualitative and quantitative tests, CD4+T lymphocyte count, HIV drug resistance tests, etc. The HIV-1/2 antibody tests are the gold standard for the diagnosis of HIV infection, and the HIV nucleic acid tests (both qualitative and quantitative) are also used for the diagnosis of HIV infection. The HIV antibody tests comprise screening tests and supplementary tests. The HIV supplementary tests comprise supplementary antibody tests (confirmation tests for antibodies) and supplementary nucleic acid tests (qualitative and quantitative nucleic acid tests). HIV nucleic acid quantification and CD4+ T lymphocyte count are two important indicators for assessment of disease progression, clinical medication, efficacy and prognosis. HIV drug resistance tests can guide the selection and replacement of ART regimens.

Many laboratory tests (e.g., polymerase chain reaction (PCR)-based test) for HIV-1 focus on the genome of HIV-1 group M. It is difficult to amplify all genotypes by conventional fluorescence quantitative PCR detection with only one set of primers and probes. Only several main types such as A, B, C, BC, AE can be detected with only one set of primers and probes. As a result, false negative or inaccurate quantification for respective subtypes may occur, and especially, group O viruses are more prone to false negative. The sensitivity of the current HIV-1 RNA fluorescence quantitative PCR reagents is greater than or equal to 50 IU/mL. Viral loads at low concentrations cannot be accurately detected and quantified. A same kind of reagent with excellent performance is the fully enclosed diagnostic system produced by Roche and called “Roche Cobas6800/8800 TaqMan HIV-1 assay”. The HIV-RNA in clinical sample can be quantified by extracting RNA using the principle of magnetic bead method in the fully automated instrument, then automatically adding sample and carrying out fluorescence PCR amplification of RNA. The diagnostic system has the advantages of high automation, simple operation and high sensitivity for detection (greater than 22 IU/mL), but may brought great financial pressure to the construction of medical and health system in China due to the closed system and the high cost of the reagents and consumables.

There is a need in the art for a HIV-1 detection reagent which enables more accurate detection of HIV-1 with high sensitivity and low cost and without false negative.

### SUMMARY

The present disclosure provides compositions methods to amplify and determine HIV-1 nucleic acid sequences by use a set of primers and probes.

In an aspect, provided herein is a composition for amplifying and detecting human immunodeficiency virus type 1 (HIV-1) nucleic acid sequence, comprising: (a) a first set comprising a first forward primer, a first reverse primer, and a first single-stranded detection probe comprising a first label, wherein the first set amplifies and detects a first portion of the HIV-1 nucleic acid sequence, wherein the first portion is gag gene; (b) a second set comprising a second forward primer, a second reverse primer, and a second single stranded detection probe comprising a second label, wherein the second set amplifies and detects a second portion of the HIV-1 nucleic acid sequence, wherein the second portion is pol gene; and (c) a third forward primer, wherein the third forward primer amplifies a third portion of the HIV-1 nucleic acid sequence, wherein the third portion is a variant region of the HIV-1 nucleic acid sequence of pol gene.

In some embodiment, the composition further comprises (d) an internal standard set comprising an internal standard forward primer, an internal standard revere primer, and a single-stranded internal standard detection probe, wherein the internal standard set amplifies and detects an internal standard if the internal standard is present. In some embodiments, the internal standard is an exogenous non-human genomic internal standard, an exogenous synthetic internal standard, or an endogenous human genomic internal standard. In some embodiments, the exogenous non-human genomic internal standard comprises a plant nucleic acid molecule, a fungi nucleic acid molecule, a bacteria nucleic acid molecule, an archaea nucleic acid molecule, or a virus nucleic acid molecule that is neither HIV-1 nor HIV type 2. In some embodiments, the exogenous non-human genomic internal standard is papillomavirus, Arabidopsis Scc3 homologue (AtSCC3), or histocompatibility complex class II beta chain paralogue (HLA-DRB3). In some embodiments, the endogenous human genomic internal standard is ribonuclease P (RnaseP), glyceraldehyde 3-phosphate dehydrogenase (GAPDH), β-actin, or 18S ribosomal ribonucleic acid (18S rRNA). In some embodiments, the first forward primer is at least 80% identical to a sequence selected from the group consisting of SEQ ID NOs: 1, 8 and 15; the first reverse primer is at least 80% identical to a sequence selected from the group consisting of SEQ ID NOs: 2, 9 and 16; the first detection probe is at least 80% identical to a sequence selected from the group consisting of SEQ ID NOs: 3, 10 or 17; the second forward primer is at least 80% identical to a sequence selected from the group consisting of SEQ ID NOs: 4, 11 or 18; the second reverse primer is at least 80% identical to a sequence selected from the group consisting of SEQ ID NOs: 5, 12 or 19; and the second detection probe is at least 80% identical to a sequence selected from the group consisting of SEQ ID NOs: 7, 14 or 21. In some embodiments, the third forward primer is at least 80% identical to a sequence selected from the group consisting of SEQ ID NOs: 6, 13 or 20. In some embodiments, the first detection probe comprises a first detectable label, and wherein the second detection probe comprises a second detectable label. In some embodiments, the first detectable label is FAM, HEX, ROX, or CY5; and wherein the second detectable label is FAM, HEX, ROX, or CY5. In some embodiments, the first detection label is different from the second detection label.

In another aspect, provided herein is a kit for amplifying and detecting human immunodeficiency virus type 1 (HIV-1) nucleic acid sequence, comprising: (a) a first set comprising a first forward primer, a first reverse primer, and a first single-stranded detection probe comprising a first label, wherein the first set amplifies and detects a first portion of the HIV-1 nucleic acid sequence, wherein the first portion is gag gene; (b) a second set comprising a second forward primer, a second reverse primer, and a second single stranded detection probe comprising a second label, wherein the second set amplifies and detects a second portion of the HIV-1 nucleic acid sequence, wherein the second portion is pol gene; (c) a third forward primer, wherein the third forward primer amplifies a third portion of the HIV-1 nucleic acid sequence, wherein the third portion is a variant region of the HIV-1 nucleic acid sequence of pol gene; (d) reagents for amplifying and detecting nucleic acid sequences; and (e) instructions for use.

In some embodiment, the kit further comprises (f) an internal standard set comprising an internal standard forward primer, an internal standard revere primer, and a single-stranded internal standard detection probe, wherein the internal standard set amplifies and detects an internal standard if the internal standard is present. In some embodiments, the first forward primer is at least 80% identical to a sequence selected from the group consisting of SEQ ID NOs: 1, 8 and 15; the first reverse primer is at least 80% identical to a sequence selected from the group consisting of SEQ ID NOs: 2, 9 and 16; the first detection probe is at least 80% identical to a sequence selected from the group consisting of SEQ ID NOs: 3, 10 or 17; the second forward primer is at least 80% identical to a sequence selected from the group consisting of SEQ ID NOs: 4, 11 or 18; the second reverse primer is at least 80% identical to a sequence selected from the group consisting of SEQ ID NOs: 5, 12 or 19; and the second detection probe is at least 80% identical to a sequence selected from the group consisting of SEQ ID NOs: 7, 14 or 21.

In still another aspect, disclosed herein is a method for detecting human immunodeficiency virus-1 (HIV-1) in a sample suspected of containing HIV-1, the method comprising: (i) contacting a sample with the composition of claim 1 and reagents for amplification and detection of nucleic acid sequences; (ii) amplifying at least a portion of the gag gene present in the sample, thereby providing a first amplified sequence, and/or amplifying at least a portion of the pol gene present in the sample, thereby providing a second amplified sequence; (iii) hybridizing the first single-stranded detection probe to the first amplified sequence, and/or hybridizing the second single-stranded detection probe to the second amplified sequence; and (iv) detecting a first signal from hybridization of the first single-stranded detection probe to the first amplified sequence, and/or detecting a second signal from hybridization of the second single-stranded detection probe to the second amplified sequence.

In some embodiments, the method further comprises: in (ii), amplifying at least a portion of the variant region present in the sample, thereby providing a third amplified sequence; in (iii), hybridizing the second single-stranded detection probe to the third amplified sequence; and in (iv), detecting a third signal from hybridization of the second single-stranded detection probe to the third amplified sequence. In some embodiments, a presence of the third amplified sequence is indicative of a presence of HIV-1 group O in the sample, and wherein absence of the third amplified sequence is indicative of an absence of HIV-1 group O in the sample. In some embodiments, the method further comprises: [in (i), contacting the sample with an internal standard set comprising an internal standard forward primer, an internal standard revere primer, and a single-stranded internal standard detection probe, wherein the internal standard set amplifies and detects an internal standard if the internal standard is present; in (ii), amplifying at least a portion of the internal standard if present, thereby providing a fourth amplified sequence; in (iii), hybridizing the single-stranded internal standard detection probe to the fourth amplified sequence; and in (iv), detecting a fourth signal from hybridization of the single-stranded internal standard detection probe to the fourth amplified sequence. In some embodiments, the sample comprises blood, serum, plasma, saliva, urine, vaginal fluid, or semen. In some embodiments, a presence of either the first amplified sequence or the second amplified sequence is indicative of a presence of HIV-1 group M in the sample, and wherein absence of both the first amplified sequence and the second amplified sequence is indicative of an absence of HIV-1 group M in the sample.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “figure” and “FIG.” herein), of which:

FIG. 1 shows one example of the detection sensitivity of the composition of the present application;

FIG. 2 shows another example of the detection sensitivity of the composition of the present application;

FIG. 3 shows the detection of clinical samples using a single set of primers and probe, HIV-ZF1/R1/P1;

FIG. 4 shows the detection of clinical samples using a single set of primers and probe, Z-POL-F1/R1/P1;

FIG. 5 shows one example of the detection of clinical samples using two sets of primers and probes, HIV-ZF1/R1/P1 and Z-POL-F1/R1/P1;

FIG. 6 shows the detection of a single clinical sample using different combinations of primers and probes;

FIG. 7 shows detection of a reference sample using two sets of primers and probes with the primer of SEQ ID NO: 6;

FIG. 8 shows detection of a reference sample using two sets of primers and probes without the primer of SEQ ID NO: 6;

FIG. 9 shows another example of the detection of clinical samples using two sets of primers and probes, HIV-ZF1/R1/P1 and Z-POL-F1/R1/P1;

FIG. 10 shows still another example of the detection of clinical samples using two sets of primers and probes, HIV-ZF2/R2/P2 and Z-POL-F2/R2/P2;

FIG. 11 shows an example of the detection of clinical samples using two sets of primers and probes, HIV-ZF3/R3/P3 and Z-POL-F3/R3/P3;

FIG. 12 shows the detection of the same clinical sample using different two sets of primers and probes;

FIG. 13 shows the detections using different internal standards.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

As used herein, the singular forms “a” “an”, and “the” include plural references unless the context clearly dictates otherwise.

As used herein, the term “PCR” generally refers to a polymerase chain reaction (PCR) process that can be used for the qualitative and quantitative determination of nucleic acid sequences. PCR can involve the measurement of the amount of amplification product (or amplicon) as a function of amplification cycle, and use such information to determine the amount of the nucleic acid sequence corresponding to the amplicon that was present in the original sample.

As used herein, the term “probe” generally refers to a molecular species or other marker that can bind to a specific target nucleic acid sequence. A probe can be any type of molecule or particle. Probes can comprise molecules and can be bound to the substrate or other solid surface, directly or via a linker molecule.

As used herein, the term “mutation” generally refers to genetic mutations or sequence variations such as a point mutation, a single nucleotide polymorphism (SNP), an insertion, a deletion, a substitution, a transposition, a translocation, a copy number variation, or another genetic mutation, alteration or sequence variation.

As used herein, the term “label” generally refers to a specific molecular structure that can be attached to a target molecule, to make the target molecule distinguishable and traceable by providing a unique characteristic not intrinsic to the target molecule.

A quantitative PCR or Q-PCR process can be described in the following non-limiting example. A PCR reaction is carried out with a pair of primers designed to amplify a given nucleic acid sequence in a sample. The appropriate enzymes and nucleotides, such as deoxynucleotide triphosphates (dNTPs), are added to the reaction, and the reaction is subjected to a number of amplification cycles. The amount of amplicon generated from each cycle is detected, but in the early cycles, the amount of amplicon can be below the detection threshold. The amplification may be occurring in two phases, an exponential phase, followed by a non-exponential plateau phase. During the exponential phase, the amount of PCR product approximately doubles in each cycle. As the reaction proceeds, however, reaction components are consumed, and ultimately one or more of the components becomes limiting. At this point, the reaction slows and enters the plateau phase. Initially, the amount of amplicon remains at or below background levels, and increases are not detectable, even though amplicon product accumulates exponentially. Eventually, enough amplified product accumulates to yield a detectable signal. The cycle number at which this occurs is called the threshold cycle, or Cₜ or CT. Since the Cₜ value is measured in the exponential phase when reagents are not limited, Q-PCR can be used to calculate the initial amount of template present in the reaction. The Cₜ of a reaction may be determined mainly by the amount of nucleic acid sequence corresponding to amplicon present at the start of the amplification reaction. If a large amount of template is present at the start of the reaction, relatively few amplification cycles may be required to accumulate enough products to give a signal above background. Thus, the reaction may have a low, or early, Cₜ. In contrast, if a small amount of template is present at the start of the reaction, more amplification cycles may be required for the fluorescent signal to rise above background. Thus, the reaction may have a high, or late, Cₜ. Methods and systems provided herein allow for the measurement of the accumulation of multiple amplicons in a single fluid in a single amplification reaction, and thus the determination of the amount of multiple nucleic acid sequences in the same sample with the methodology of Q-PCR described above.

As used herein, the “international unit” or “IU” for HIV-1 RNA generally is equivalent to 0.35 copies of HIV-1 RNA for the Third HIV-1 World Health Organization (WHO) International Standard. One genome equivalent (ge) is equal to about 1.7 IU.

As used herein, the term “nucleotide” generally refers to a molecule that can serve as the monomer, or subunit, of a nucleic acid, such as deoxyribonucleic acid (DNA) or ribonucleic acid (RNA). A nucleotide may be a deoxynucleotide triphosphate (dNTP) or an analog thereof (e.g., a molecule having a plurality of phosphates in a phosphate chain, such as 2, 3, 4, 5, 6, 7, 8, 10, or more phosphates). A nucleotide may generally include adenosine (A), cytosine (C), guanine (G), thymine (T) and uracil (U), or variants thereof. A nucleotide may include any subunit that can be incorporated into a growing nucleic acid strand. Such subunit can be an A, C, G, T, or U, or any other subunit that is specific to one or more complementary A, C, G, T, or U, or complementary to a purine (e.g., A or G, or variant thereof) or a pyrimidine (e.g., C, T, or U, or variant thereof). A subunit can enable individual nucleic acid bases of group of bases (e.g., AA, TA, AT, GC, CG, CT, TC, GT, TC, AC, CA, or uracil counterparts thereof) to be resolved. A nucleotide may be labeled or unlabeled. A labeled nucleotide may yield a detectable signal, such as an optical, electrostatic, or electrochemical signal.

As used herein, the terms “polynucleotide,” “oligonucleotide,” “nucleotide,” “nucleic acid,” and “nucleic acid molecule” generally refer to a polymeric form of nucleotides (polynucleotides) of various lengths, either ribonucleotide (RNA) or deoxyribonucleotides (DNA). Examples of nucleotide sequences are sequences corresponding to natural or synthetic RNA or DNA including genomic DNA and messenger RNA. The length of the sequence can be any length that can be amplified into nucleic acid amplification products, or amplicons, for example, up to about 20, 40, 100, 200, 300, 400, 500, 600, 00, 800, 21000, 1200, 1500, 2000, 5000, 12000, or more than 10000 nucleotides in length.

The compositions, kits, and methods described herein may be used for simultaneously performing a plurality of reactions (e.g., a biochemical reaction, a chemical reaction) and real-time monitoring the progress of the reactions via, for example, detecting and/or determining the presence or absence, amount, quantity, concentration, activity and/or binding characteristics of one or more target substances (e.g., HIV-1 nucleic acid sequence or derivatives thereof) in a reaction chamber. The amount, quantity, concentration, activity and/or binding characteristics of target substances may be monitored and/or determined by detecting signals produced upon the occurrence of binding between the target substances and the probes. With provided methods and systems of the present disclosure, the presence or absence of the target substances may be determined with high sensitivity and/or specificity. For example, the presence or absence of a target analyte may be determined or detected at a sensitivity of at least about 50%, 60%, 70%, 80%, 85%, 90%, 91%, 92%, 93%, 94%, 95%, 96%, 97%, 98%, 99%, 99.9%, 99.99%, 99.999%, or 99.9999%. Similarly, the sensitivity of the methods provided herein may be at least about 50%, 60%, 70%, 80%, 85%, 90%, 91%, 92%, 93%, 94%, 95%, 96%, 97%, 98%, 99%, 99.9%, 99.99%, 99.999%, or 99.9999%.

### Nucleic Acid Amplification

Compositions, kts and methods provided herein can be used for performing nucleic acid amplification reaction on a plurality of nucleotide sequences in a fluid. The presence, quantity and/or binding activity of the amplified products (i.e., amplicons) may be simultaneously detected and monitored in real-time as the reaction proceeds. Methods of amplification may include, for example, polymerase chain reaction (PCR).

The amplification method can be temperature cycling or be isothermal. The amplification method can be exponential or linear. For amplifications with temperature cycling, a temperature cycle may generally correspond to an amplification cycle. Isothermal amplifications can in some cases have amplification cycles, such as denaturing cycles, and in other cases, the isothermal amplification reaction will occur monotonically without any specific amplification cycle.

The amplification method may be used to amplify specific regions (i.e., target regions), or nucleotide sequences of a nucleic acid molecule (e.g., DNA, RNA). This region can be, for example, a single gene, a part of a gene, or a non-coding sequence.

The amplification method may comprise: (1) a template that contains the region of the nucleic acid sequence to be amplified; (2) one or more primers, which are complementary to the target region at the 5′ and 3′ ends of the region that is to be amplified; (3) a polymerase (e.g. Taq polymerase), used to synthesize a copy of the region to be amplified; (4) deoxynucleotide triphosphates (dNTPs); (5) a buffer solution, which provides a suitable chemical environment for optimum activity and stability of the polymerase; and/or (6) a divalent cation such as magnesium or manganese ions.

A primer may be a nucleic acid strand, or a related molecule that serves as a starting point for nucleic acid replication. A primer may often be required because some nucleic acid polymerases cannot begin synthesizing a new strand from scratch, but can only add to an existing strand of nucleotides. The length of the primers may vary. Primers with longer or shorter lengths may be used, dependent upon, the application. For example, in some cases, chemically synthesized DNA molecules with a length about 10 to about 30 bases may be used as primers. In some cases, the length of primers can be for example about 20-30 nucleotides, and the sequence of the primers are complementary to the beginning and the end of the target fragment to be amplified. The primers may anneal (adhere) to the template at these starting and ending points, where polymerase binds and begins the synthesis of the new strand. In some cases, degenerate primers may be used. Degenerate primers comprise mixtures of similar, but not identical, primers.

As provided in the present disclosure, primers or probes may or may not be labeled with a reporter molecule (e.g., a label). The reporter molecule may be configured to facilitate and/or enable the detection and/or monitoring of the presence, quantity, concentration or binding activity of the primers, amplicons, or the probes. The reporter molecules can be optical, electrical or electrochemical. Examples of reporter molecules may include, but not limited to fluorescent, quenchers, fluorophores, members of a fluorescence resonance energy transfer (FRET) pair, redox species, or combinations thereof.

A wide variety of fluorescent molecules (e.g., small molecules, fluorescent proteins and quantum dots) can be utilized in the present disclosure. Non-limiting examples of fluorescent molecules (or fluorophores) may include: 1,5 IAEDANS; 1,8-ANS; 4-Methylumbelliferone; 5-carboxy-2,7-dichlorofluorescein; 5-Carboxyfluorescein (5-FAM); 5-Carboxynapthofluorescein; 5-Carboxytetramethyirhodamine (5-TAMRA); 5-FAM (5-Carboxyfluorescein); 5-HAT (Hydroxy Tryptamine); 5-Hydroxy Tryptamine (HAT); 5-ROX (carboxy-X-rhodamine); 5-TAMRA (5-Carboxytetramethylrhodamine); 6-Carboxyrhodamine 6G; 6-CR 6G; 6-JOE; 7-Amino-4-methylcoumarin; 7-Aminoactinomycin D (7-AAD); 7-Hydroxy-4-methylcoumarin; 9-Amino-6-chloro-2-methoxyacridine; ABQ; Acid Fuchsin; ACMA (9-Amino-6-chloro-2-methoxyacridine); Acridine Orange; Acridine Red; Acridine Yellow; Acriflavin; Acriflavin Feulgen SITSA; Aequorin (Photoprotein); AFPs—AutoFluorescent Protein—(Quantum Biotechnologies); Alexa Fluor 350™; Alexa Fluor 430™; Alexa Fluor 488™; Alexa Fluor 532™; Alexa Fluor 546™; Alexa Fluor 568™. Alexa Fluor 594™; Alexa Fluor 633™; Alexa Fluor 647™; Alexa Fluor 660™; Alexa Fluor 680™. Alizarin Complexion; Alizarin Red; Allophycocyanin (APC); AMC, AMCA-S; AMCA (Aminomethylcoumarin); AMCA-X; Aminoactinomycin D; Aminocoumarin; Anilin Blue; Anthrocyl stearate; Aminomethylcoumarin (AMCA); APC (Allophycocyanin); APC-Cy7; APTRA-BTC; APTS; Astrazon Brilliant Red 4G; Astrazon Orange R; Astrazon Red 6B; Astrazon Yellow 7 GLL; Atabrine; ATTO-TAG™ CBQCA; ATTO-TAG™ FQ; Auramine; Aurophosphine G; Aurophosphine; BAO 9 (Bisaminophenyloxadiazole); BCECF (high pH); BCECF (low pH); Berberine Sulphate; Beta Lactamase; Bimane; Bisbenzamide; Bisbenzimide (Hoechst); bis-BTC; Blancophor FFG; Blancophor SV; BOBO™-1; BOBO™-3; Bodipy 492/515; Bodipy 493/503; Bodipy 500/510; Bodipy 505/515; Bodipy 530/550; Bodipy 542/563; Bodipy 558/568; Bodipy 564/570; Bodipy 576/589; Bodipy 581/591; Bodipy 630/650-X; Bodipy 650/665-X; Bodipy 665/676; Bodipy FI; Bodipy FL ATP; Bodipy FI-Ceramide; Bodipy R6G SE; Bodipy TMR; Bodipy TMR-X conjugate; Bodipy TMR-X, SE; Bodipy TR; Bodipy TR ATP; Bodipy TR-X SE; BO-PRO™-1; BO-PRO™-3; Brilliant Sulphoflavin FF; BTC; BTC-SN; Calcein; Calcein Blue; Calcium Crimson™; Calcium Green; Calcium Green-1 Ca.sup.2+Dye; Calcium Green-2 Ca.sup.2+; Calcium Green-SN Ca.sup.2+; Calcium Green-C18 Ca.sup.2.sup.+; Calcium Orange; Calcofluor White; Carboxy-X-rhodamine (5-ROX); Cascade Blue™; Cascade Yellow; Catecholamine; CCF2 (GeneBlazer); CFDA; Chlorophyll; Chromomycin A; Chromomycin A; CL-NERF; CMFDA; Coumarin Phalloidin; C-phycocyanine; CPM Methylcoumarin; CTC; CTC Formazan; Cy2™; Cy3.1 8; Cy3.5™; Cy3™; Cy5.1 8; Cy5.5™; Cy5™; Cy7™; cyclic AMP Fluorosensor (FiCRhR); Dabcyl; Dansyl; Dansyl Amine; Dansyl Cadaverine; Dansyl Chloride; Dansyl DUPE; Dansyl fluoride; DAPI; Dapoxyl; Dapoxyl 2; Dapoxyl 3′ DCFDA; DCFH (Dichlorodihydrofluorescein Diacetate); DDAO; DHR (Dihydrorhodamine 123); Di-4-ANEPPS; Di-8-ANEPPS (non-ratio); DiA (4-Di-16-ASP); Dichlorodihydrofluorescein Diacetate (DCFH); DiD-Lipophilic Tracer; DiD (DiIC18(5)); DIDS; Dihydrorhodamine 123 (DHR); Dil (DiIC18(3)); Dinitrophenol; DiO (DiOC18(3)); DiR; DiR (DiIC18(7)); DM-NERF (high pH); DNP; Dopamine; DTAF; DY-630-NHS; DY-635-NHS; ELF 97; Eosin; Erythrosin; Erythrosin ITC; Ethidium Bromide; Ethidium homodimer-1 (EthD-1); Euchrysin; EukoLight; Europium (III) chloride; EYFP; Fast Blue; FDA; Feulgen (Pararosaniline); FIF (Formaldehyde Induced Fluorescence); FITC; Flazo Orange; Fluo-3; Fluo-4; Fluorescein (FITC); Fluorescein Diacetate; Fluoro-Emerald; Fluoro-Gold (Hydroxystilbamidine); Fluor-Ruby; Fluor X; FM 1-43™; FM 4-46; Fura Red™ (high pH); Fura Red™/Fluo-3; Fura-2; Fura-2/BCECF; Genacryl Brilliant Red B; Genacryl Brilliant Yellow 10GF; Genacryl Pink 3G; Genacryl Yellow 5GF; GeneBlazer (CCF2); Gloxalic Acid; Granular blue; Haematoporphyrin; Hoechst 33258; Hoechst 33342; Hoechst 34580; HPTS; Hydroxycoumarin; Hydroxystilbamidine (FluoroGold); Hydroxytryptamine; Indo-1, high calcium; Indo-1, low calcium; Indodicarbocyanine (DiD); Indotricarbocyanine (DiR); Intrawhite Cf; JC-1; JO-JO-1; JO-PRO-1; LaserPro; Laurodan; LDS 751 (DNA); LDS 751 (RNA); Leucophor PAF; Leucophor SF; Leucophor WS; Lissamine Rhodamine; Lissamine Rhodamine B; Calcein/Ethidium homodimer; LOLO-1; LO-PRO-1; Lucifer Yellow; Lyso Tracker Blue; Lyso Tracker Blue-White; Lyso Tracker Green; Lyso Tracker Red; Lyso Tracker Yellow; LysoSensor Blue; LysoSensor Green; LysoSensor Yellow/Blue; Mag Green; Magdala Red (Phloxin B); Mag-Fura Red; Mag-Fura-2; Mag-Fura-5; Mag-indo-1; Magnesium Green; Magnesium Orange; Malachite Green; Marina Blue; Maxilon Brilliant Flavin 10 GFF; Maxilon Brilliant Flavin 8 GFF; Merocyanin; Methoxycoumarin; Mitotracker Green FM; Mitotracker Orange; Mitotracker Red; Mitramycin; Monobromobimane; Monobromobimane (mBBr-GSH); Monochlorobimane; MPS (Methyl Green Pyronine Stilbene); NBD; NBD Amine; Nile Red; Nitrobenzoxadidole; Noradrenaline; Nuclear Fast Red; Nuclear Yellow; Nylosan Brilliant lavin E8G; Oregon Green; Oregon Green 488-X; Oregon Green™; Oregon Green™ 488; Oregon Green™ 500; Oregon Green™ 514; Pacific Blue; Pararosaniline (Feulgen); PBFI; PE-Cy5; PE-Cy7; PerCP; PerCP-Cy5.5; PE-TexasRed [Red 613]; Phloxin B (Magdala Red); Phorwite AR; Phorwite BKL; Phorwite Rev; Phorwite RPA; Phosphine 3R; PhotoResist; Phycoerythrin B [PE]; Phycoerythrin R [PE]; PKH26 (Sigma); PKH67; PMIA; Pontochrome Blue Black; POPO-1; POPO-3; PO-PRO-1; PO-PRO-3; Primuline; Procion Yellow; Propidium lodid (PL); PyMPO; Pyrene; Pyronine; Pyronine B; Pyrozal Brilliant Flavin 7GF; QSY 7; Quinacrine Mustard; Red 613 [PE-TexasRed]; Resorufin; RH 414; Rhod-2; Rhodamine; Rhodamine 110; Rhodamine 123; Rhodamine 5 GLD; Rhodamine 6G; Rhodamine B; Rhodamine B 200; Rhodamine B extra; Rhodamine BB; Rhodamine BG; Rhodamine Green; Rhodamine Phallicidine; Rhodamine Phalloidine; Rhodamine Red; Rhodamine WT; Rose Bengal; R-phycocyanine; R-phycoerythrin (PE); S65A; S65C; S65L; S65T; SBFI; Serotonin; Sevron Brilliant Red 2B; Sevron Brilliant Red 4G; Sevron Brilliant Red B; Sevron Orange; Sevron Yellow L; SITS; SITS (Primuline); SITS (Stilbene Isothiosulphonic Acid); SNAFL calcein; SNAFL-1; SNAFL-2; SNARF calcein; SNARF1; Sodium Green; SpectrumAqua; SpectrumGreen; SpectrumOrange; Spectrum Red; SPQ (6-methoxy-N-(3-sulfopropyl)quinolinium); Stilbene; Sulphorhodamine B can C; Sulphorhodamine Extra; SYTO 11; SYTO 12; SYTO 13; SYTO 14; SYTO 15; SYTO 16; SYTO 17; SYTO 18; SYTO 20; SYTO 21; SYTO 22; SYTO 23; SYTO 24; SYTO 25; SYTO 40; SYTO 41; SYTO 42; SYTO 43; SYTO 44; SYTO 45; SYTO 59; SYTO 60; SYTO 61; SYTO 62; SYTO 63; SYTO 64; SYTO 80; SYTO 81; SYTO 82; SYTO 83; SYTO 84; SYTO 85; SYTOX Blue; SYTOX Green; SYTOX Orange; Tetracycline; Tetramethylrhodamine (TRITC); Texas Red™; Texas Red-X™ conjugate; Thiadicarbocyanine (DiSC3); Thiazine Red R; Thiazole Orange; Thioflavin 5; Thioflavin S; Thioflavin TCN; Thiolyte; Thiozole Orange; Tinopol CBS (Calcofluor White); TMR; TO-PRO-1; TO-PRO-3; TO-PRO-5; TOTO-1; TOTO-3; TriColor (PE-Cy5); TRITC TetramethylRodaminelsoThioCyanate; True Blue; TruRed; Ultralite; Uranine B; Uvitex SFC; WW 781; X-Rhodamine; XRITC; Xylene Orange; Y66F; Y66H; Y66W; YO-PRO-1; YO-PRO-3; YOYO-1; YOYO-3, Sybr Green, Thiazole orange (interchelating dyes), Alexa Fluor dye series (e.g., Alexa Fluor 350, Alexa Fluor 405, 430, 488, 500, 514, 532, 546, 555, 568, 594, 610, 633, 635, 647, 660, 680, 700, and 750), Cy Dye fluorophore series (e.g., Cy3, Cy3B, Cy3.5, Cy5, Cy5.5, Cy7), Oyster dye fluorophores (e.g., Oyster-500, -550, -556, 645, 650, 656), DY-Labels series (e.g., DY-415, -495, -505, -547, -548, -549, -550, -554, -555, -556, -560, -590, -610, -615, -630, -631, -632, -633, -634, -635, -636, -647, -648, -649, -650, -651, -652, -675, -676, -677, -680, -681, -682, -700, -701, -730, -731, -732, -734, -750, -751, -752, -776, -780, -781, -782, -831, -480XL, -481XL, -485XL, -510XL, -520XL, -521XL), ATTO fluorescent labels (e.g., ATTO 390, 425, 465, 488, 495, 520, 532, 550, 565, 590, 594, 610, 611X, 620, 633, 635, 637, 647, 647N, 655, 680, 700, 725, 740), CAL Fluor and Quasar dyes (e.g., CAL Fluor Gold 540, CAL Fluor Orange 560, Quasar 570, CAL Fluor Red 590, CAL Fluor Red 610, CAL Fluor Red 635, Quasar 670), quantum dots (e.g., Qdot 525, Qdot565, Qdot585, Qdot605, Qdot655, Qdot705, Qdot 800), fluorescein, rhodamine, phycoerythrin, or combinations thereof.

### Primers/Probe Set

Accordingly, in a first aspect, the present application provides a composition for detecting HIV-1 comprising:

A)
a forward primer HIV-ZF1:
(SEQ ID NO: 1)
5′-CCTGGGAGCTCTCTGGCTA-3′,
a reverse primer HIV-ZR1:
(SEQ ID NO: 2)
5′-AAGCACTCAAGGCAAGCTTTAT-3′,
a detection probe HIV-ZP1:
(SEQ ID NO: 3)
5′-AGGCTTAAGCAGTGGGTTCC-3′,
a forward primer Z-POL-F1:
(SEQ ID NO: 4)
5′-AAATCACTCTTTGGCARCGAC-3′,
a reverse primer Z-POL-R1:
(SEQ ID NO: 5)
5′-CCCCCTATCATTTTTGGTTTC-3′,
a forward primer Z-POL-FO1:
(SEQ ID NO: 6)
5′-ATCCCTCTTTGGGACAGACC-3′,
and
a detection probe Z-POL-P1:
(SEQ ID NO: 7)
5′-ACTGTATCATCTGCYCCTGT-3′;
or
B)
a forward primer HIV-ZF2:
(SEQ ID NO: 8)
5′-GAGCCTGGGAGCTCTCTGGC-3′,
a reverse primer HIV-ZR2:
(SEQ ID NO: 9)
5′-GCACTCAAGGCAAGCTTTATTGA-3′,
a detection probe HIV-ZP2:
(SEQ ID NO: 10)
5′-GCTTAAGCAGTGGGTTCCCTAG-3′,
a forward primer Z-POL-F2:
(SEQ ID NO: 11)
5′-CTCAGGTCACTCTTTGGCAACG-3′,
a reverse primer Z-POL-R2:
(SEQ ID NO: 12)
5′-TCCTCCTATCATTTTTGGTTTCC-3′,
a forward primer Z-POL-FO2:
(SEQ ID NO: 13)
5′-CAAATCCCTCTTTGGGACAGAC-3′,
and
a detection probe Z-POL-P2:
(SEQ ID NO: 14)
5′-GTATCATCTGCTCCTGTATCT-3′;
or
C)
a forward primer HIV-ZF3:
(SEQ ID NO: 15)
5′-TGGGAGCTCTCTGGCTAACTA-3′,
a reverse primer HIV-ZR3:
(SEQ ID NO: 16)
5′-CTTGAAGCACTCAAGGCAAGCTTTA-3′,
a detection probe HIV-ZP3:
(SEQ ID NO: 17)
5′-ATTGAGGCTTAAGCAGTGGGT-3′,
a forward primer Z-POL-F3:
(SEQ ID NO: 18)
5′-GTCACTCTTTGGCAACGACCCCT-3′,
a reverse primer Z-POL-R3:
(SEQ ID NO: 19)
5′-CCCCTATCATTTTTGGTTTCCATC-3′,
a forward primer Z-POL-FO3:
(SEQ ID NO: 20)
5′-TCCCTCTTTGGGACAGACCAR-3′,
and
a detection probe Z-POL-P3:
(SEQ ID NO: 21)
5′-AATACTGTATCATCTGCTCCTG-3′.

HIV-1 can be detected using the composition of the present application with a sensitivity of 22 IU/mL. HIV-1 can be detected by using primers/probe set having 80% identity to the corresponding primers/probe set disclosed in the present application. Moreover, two sets of primers and probes for two different regions (gag and pol) of HIV-1 group M and an additional forward primer for one region (pol) of HIV-1 group O are combined in the composition of the present application to prepare a reaction system, and the HIV-1 RNA are detected using the reaction system to identify a positive reaction, effectively reducing the probability of false negative, especially, of group O viruses. There is no need for a fully enclosed diagnostic system, which effectively reduces costs.

Further, the above detection probes have fluorescent groups which are different from each other and non-interfering with each other.

Further, the above detection probes have fluorescent groups which may be the same.

Herein, “different from each other and non-interfering with each other” means that the fluorescent groups used in each set of the probes in the composition are different and will not affect the detection using each other, that is, their detection can be performed in different channels. For example, FAM, HEX, ROX and CY5 do not have close absorbance values and thus different channels can be selected without interference with each other.

Further, the composition includes an internal standard forward primer, an internal standard reverse primer, and an internal standard probe, for monitoring.

Further, the internal standard includes an exogenous non-human genomic internal standard, exogenous synthetic internal standard, or endogenous human genomic internal standard.

In a specific embodiment, the exogenous non-human genomic internal standard sequence (partial sequence of papillomavirus from the family Papovaviridae) is:

(SEQ ID NO: 22)
5′-TTGTAAGTGTGAAGCCAGAATTGAGCTAGTAGTAGAAAGCTCAGCA
GACGACCTTCGAGCATTCCAGCAGCTGTTTCTGAACACCCTGTCCTTTG
TGTGTCCGTGGTGAAGAGTTTATTAATGAAGATATTGTAAATTTGCCCG
AGAATGTCGATTGGAGAAAAAAAGGAGCTGTCACTCCCGTAAAACATCA
GGGTTCATGCGGTAGTTGTTGGGCATTCTCGGCCGTTGCAACTGTAGAG
GGAATAAATAAGATTAGAACTGGAAAATTAGTAGAATTATCAGAGCAAG
AACTTGTTGACTGTGAAAGACGTAGCCATGGGGCAAAGACCGTTGTGTC
CAGAAGAAAAACAAAGACATTTGGATAAAAAGAAACGATTCCACAACAT
AGGAGGAAGGTGGACAGGACGTTGCATAGCATGTTGGAGAAGACCTCGT
ACTGAAACCCAAGTGTAAACATGCGATATTTTATACT-3.

In a specific embodiment, the exogenous synthetic internal standard sequence is:

(SEQ ID NO: 23)
5′-TTCATACTCATGGCGAAGTACGCAACAAGTCGAACCCTCAATCCC
AGTGAGGACTATTGGGGGTTACTTCGCAAGGACGCCGCATGTGCAGTT
CCTCAGTCTTCCTTCTCCTCCATATGAAGATTTATCCCATACGGTGCT
AGCGAAGCAAGTCTTCCGAGTTCACATCTCGAACGAAGGTCACTCACA
GTTCCGAAGGTCACTACGGTTTCCGAGTCTTATTGTTTCTTCCACGTA
TGACAGATACCCTCAACCCTCAGGCCCACGGGGGGTTGCTACGCCCAG
GGCTACTCCGTTGGCCCTGAAGGGTTACTTCGTTAGGGCTACTTGGAG
GAGTTGACCAACCCGAACATGGTGCCTTGATCTTCAGCTTTCCAATAA
GAAGATCATTGATGCAGCACACTGAGCTAAATTCTTAAACTATCTTTA
CCGGTGTGCTGCCTCGCAGAGATTCTCAACCTAGTCCGCGCATACTCT
TTCCGTTTCTGTCTTACAGTCTTTCTTGTTCTTCTTTTCCCTATAGTT
AGAAGATGAGCCCCTGGGGTGCCCTGCCCTTCTTTTACGGCTGATGGA
CATTCAATAGATTTATCTGCTGTTGCCTGATCTACGACCCCCTCAATC
TGTCGAAGCAAATGCTGCACAGAGGGAGAAGTTGCCTGACCGGGCTAT
TCCCTTGTTATCTGTTGTCCAACTGGTTGAATAGTAATCTGTGCTGGA
TCCCTCTGAAGCTGGTCATTC-3′.

In a specific embodiment, the endogenous human genomic internal standard sequence (partial sequence of RNaseP RNA) is:

(SEQ ID NO: 24)
5′-ATAGGGCGGAGGGAAGCTCATCAGTGGGGCCACGAGCTGAGTGCGT
CCTGTCACTCCACTCCCATGTCCCTTGGGAAGGTCTGAGACTAGGGCCA
GAGGCGGCCCTAACAGGGCTCTCCCTGAGCTTCGGGGAGGTGAGTTCCC
AGAGAACGGGGCTCCGCGCGAGGTCAGACTGGGCAGGAGATGCCGTGGA
CCCCGCCCTTCGGGGAGGGGCCCGGCGGATGCCTCCTTTGCCGGAGCTT
GGAACAGACTCACGGCCAGCGAAGTGAGTTCAATGGCTGAGGTGAGGTA
CCCCGCAGGGGACCTCATAACCCAATTCAGACTACTCTCCTCCGCCCAT
T-3′.

In a specific embodiment, the composition further includes: (A) an exogenous non-human genomic internal standard forward primer as set forth in HPGT31-F: 5′-CAAAGACCGTTGTGTCCAGAAG-3′ (SEQ ID NO: 25), an exogenous non-human genomic internal standard reverse primer as set forth in HPGT31-R: 5′-CGCATGTTTACACTTGGGTTTC-3′ (SEQ ID NO: 26), and an exogenous non-human genomic internal standard probe as set forth in HPGT31-P: 5′-CAACGTCCTGTCCACCTTCCTCCTATG-3′ (SEQ ID NO: 27); or

(B) an exogenous synthetic internal standard forward primer as set forth in NNJ-F: 5′-AGTACGCAACAAGTCGAACC-3′ (SEQ ID NO: 28), an exogenous synthetic internal standard reverse primer as set forth in NNJ-R: 5′-TGGAGGAGAAGGAAGACTGAGGAA-3′ (SEQ ID NO: 29), and an exogenous synthetic internal standard probe as set forth in NNJ-P: 5′-CCCAGTGAGGACTACTCCGTTGGGGG-3′ (SEQ ID NO: 30); or

(C) an exogenous endogenous human genomic internal standard forward primer as set forth in RNaseP-F: 5′-GCGGAGGGAAGCTCATCAG-3′ (SEQ ID NO: 31), an endogenous human genomic internal standard reverse primer as set forth in RNaseP-R: 5′-GGACATGGGAGTGGAGTGACA-3′ (SEQ ID NO: 32), and an endogenous human genomic internal standard probe as set forth in RNaseP-P: 5′-CACGAGCTGAGTGCGTCCT-3′ (SEQ ID NO: 33).

As used herein, the term “internal standard” generally refers to a nucleic acid molecule having a non-target sequence that can be amplified along with the nucleic acid molecule having the target sequence in the same reaction chamber as an in situ quality control. The present application provides different forms of internal standards, including, for example, an exogenous non-human genomic internal standard, an exogenous synthetic internal standard, or an endogenous human genomic internal standard. For example, an exogenous non-human genomic internal standard can be selected from part of the papillomavirus sequence from the Polyomaviridae family, then design the corresponding primer set and probes and confirm their high specificity for the primers/probes through the Blast program. An exogenous synthetic internal standard can be a pseudovirus sequence that is designed and synthesized with the goal of not causing interference with the target amplicon. Both types of exogenous internal standards can be added to the nucleic acid extraction reagents in equal amount before extracting nucleic acids from the sample. The use of these exogenous internal standards can monitor the process of the sample from the extraction step to the amplification step. The endogenous human genomic internal standard can be ribonuclease P (RNaseP), which is widely present in the mitochondria and nucleoli of eukaryotes and prokaryotes, and is universally present in the cells of human organs and tissues. In this present application, a primer set and probe is designed to amplify RNaseP. If there are no issues with the sampling step and the extraction step, the amplification curve of the internal standard can be observed in the internal standard channel, simultaneously monitoring the progression of the test results of the sample.

Further, the internal standard probe and the detection probe have fluorescent groups which are different from each other and non-interfering with each other.

In the present application, the fluorescent reporter groups may be selected from, but is not limited to, FAM, HEX, ROX, VIC, CY5, 5-TAMRA, TET, CY3 and JOE.

In a specific embodiment, the fluorescent groups of the detection probes are FAM.

In a specific embodiment, the fluorescent groups of the detection probes are FAM and CY5.

In a specific embodiment, the fluorescent group of the internal standard probe is HEX.

Furthermore, the probe further has a quenching group, such as BHQ1 or BHQ2, at the 3′ end.

In a specific embodiment, the probe has BHQ1 at the 3′ end.

Further, the primers are present in the composition in an amount of 0.1-0.3 μM; and the probes are present in the composition in an amount of 0.05-0.20 μM.

In a specific embodiment, the components in the compositions of the present application are present in separate packages.

In a specific embodiment, the components in the compositions of the present application are present in a mixture.

In a second aspect, the present application provides use of the above-mentioned composition of the present application in preparing a kit for detecting HIV-1.

In a third aspect, the present application provides a kit for detecting HIV-1, comprising the above-mentioned composition of the present application.

Furthermore, the kit further includes a negative quality control and a positive quality control.

In a specific embodiment, the negative quality control is at least one of DEPC H2O, normal saline, TE buffer, inactivated human immunodeficiency virus-negative plasma, and the positive quality control is a pseudovirus containing the internal standard gene sequence, serially diluted in 0.01% TE-SDS diluent.

Further, the kit further includes a nucleic acid release system and a nucleic acid amplification system.

Further, the nucleic acid release system includes an RNA extraction solution containing magnetic beads.

Further, a washing liquid 2 in the RNA extraction solution contains silicone oil.

In a specific embodiment, the silicone oil plays the role in protecting the PCR reaction solution. Reagent evaporation and aerosol contamination can be effectively reduced during heating and amplification at the high temperature.

Further, the kit includes 4 standard products (i.e. quantitative reference) at different amounts. The standard products are a pseudovirus containing a known gene segment RNA.

An unknown sample and the standard products are subjected to reaction in the same fluorescent PCR experiment. Ct values obtained from the standard products are fitted to a standard curve to calculate the amount of the unknown sample. The negative and positive controls are provided for the operation and quantitative calculation of the kit for quality control.

Further, the kit includes at least one of dNTPs, a PCR buffer, Mn²⁺ and Mg²⁺.

Further, the kit includes at least one of a nucleic acid release agent, a nucleic acid extraction reagent, reverse transcriptase, uracil glycosylase and DNA polymerase.

Further, the kit includes at least one of a nucleic acid release reagent, a nucleic acid extraction reagent, dNTPs, reverse transcriptase, uracil glycosylase, DNA polymerase, a PCR buffer and Mg²⁺.

Further, Mn²⁺ is manganese acetate at 10-1000 mM.

Further, the reverse transcriptase is at a concentration of 5 U to 15 U per reaction, and for example, the reverse transcriptase can be murine leukemia reverse transcriptase (MMLV) or Tth enzyme; the DNA polymerase is at a concentration of 3 U to 15 U per reaction, and for example, the DNA polymerase can be Taq enzyme.

In a specific embodiment, the kit of the present application includes reverse transcriptase, Taq enzyme, uracil glycosylase, Mg²⁺, Mn²⁺, RNasin®, dNTPs, primers, probes and a PCR buffer.

Common PCR buffers are composed of buffer systems such as Tris-HCl, MgCl₂, KCl, and Triton X-100. Generally, the total volume in a single PCR reaction tube is 20 μl to 100 μl.

In a specific embodiment, the kit of the present application is compatible with a digital PCR amplification system, that is, the kit can be directly used for amplification on a digital PCR instrument.

In a fourth aspect, the present application provides a method for detecting HIV-1, comprising:
1) extracting or releasing nucleic acids from a sample to be tested;
2) subjecting the nucleic acids obtained in 1) to fluorescence quantitative PCR using the above-mentioned composition of the present application or the above-mentioned kit of the present application; and
3) obtaining a result and analyzing the result.

In the present application, the sample to be tested may be semen, vaginal discharge, tissue fluid, blood, etc., but is not limited thereto.

Further, the fluorescence quantitative PCR is performed under the following conditions:

1 cycle of reverse transcription at a temperature of 50-60° C. for 5-30 min; 1 cycle of cDNA pre-denaturation at a temperature of 95° C. for 1-10 min; and 40-50 cycles of denaturation at a temperature of 95° C. for 5-20 sec and annealing at 55-60° C. for 20-60 sec, with fluorescence collecting.

In a specific embodiment, the fluorescence quantitative PCR is performed under the following conditions: 1 cycle of reverse transcription at a temperature of 60° C. for 30 min; 1 cycle of cDNA pre-denaturation at a temperature of 95° C. for 1 min; and 45 cycles of denaturation at a temperature of 95° C. for 15 sec and annealing at 58° C. for 10 sec, with fluorescence collecting.

In a specific embodiment, there is provided a method for detecting HIV-1, comprising:
1) extracting or releasing nucleic acids from a sample to be tested;
2) subjecting the nucleic acids obtained in 1) to fluorescence quantitative PCR using the above-mentioned composition of the present application or the above-mentioned kit of the present application, and
3) obtaining a result and analyzing the result.

Further, the fluorescence quantitative PCR is performed under the following conditions:

One cycle of reverse transcription at a temperature of 50-60° C. for 5-30 min; 1 cycle of cDNA pre-denaturation at a temperature of 95° C. for 1-10 min; and 40-50 cycles of denaturation at a temperature of 95° C. for 5-20 sec and annealing at 55-60° C. for 20-60 sec, with fluorescence collecting.

In a specific embodiment, the fluorescence quantitative PCR is performed under the following conditions: 1 cycle of reverse transcription at a temperature of 60° C. for 30 min; 1 cycle of cDNA pre-denaturation at a temperature of 95° C. for 1 min; and 45 cycles of denaturation at a temperature of 95° C. for 15 sec and annealing at 58° C. for 10 sec, with fluorescence collecting.

Hereinafter, the present application will be described in detail with reference to specific embodiments and examples. The advantages and various effects of the present application will be more clearly presented therefrom. It should be understood by those skilled in the art that these specific embodiments and examples are intended to illustrate, but not to limit, the present application.

### EXAMPLES

### Example 1. Primers and Probes Used in the Present Disclosure

forward primer HIV-ZF1:
(SEQ ID NO: 1)
5′-CCTGGGAGCTCTCTGGCTA-3′,
reverse primer HIV-ZR1:
(SEQ ID NO: 2)
5′-AAGCACTCAAGGCAAGCTTTAT-3′,
detection probe HIV-ZP1:
(SEQ ID NO: 3)
5′-AGGCTTAAGCAGTGGGTTCC-3′,
forward primer Z-POL-F1:
(SEQ ID NO: 4)
5′-AAATCACTCTTTGGCARCGAC-3′,
reverse primer Z-POL-R1:
(SEQ ID NO: 5)
5′-CCCCCTATCATTTTTGGTTTC-3′,
forward primer Z-POL-FO1:
(SEQ ID NO: 6)
5′-ATCCCTCTTTGGGACAGACC-3′,
detection probe Z-POL-P1:
(SEQ ID NO: 7)
5′-ACTGTATCATCTGCYCCTGT-3′,
internal standard forward primer HPGT31-F:
(SEQ ID NO: 25)
5′-CAAAGACCGTTGTGTCCAGAAG-3′,
internal standard reverse primer HPGT31-R:
(SEQ ID NO: 26)
5′-CGCATGTTTACACTTGGGTTTC-3′,
and
internal standard probe HPGT31-P:
(SEQ ID NO: 27)
5′-CAACGTCCTGTCCACCTTCCTCCTATG-3′.

Fluorescent groups of the detection probes were FAM, and fluorescent groups of the internal standard probe were HEX.

### Example 2. Another Set of Primers and Probes Used in the Present Disclosure

forward primer HIV-ZF2:
(SEQ ID NO: 8)
5′-GAGCCTGGGAGCTCTCTGGC-3′,
reverse primer HIV-ZR2:
(SEQ ID NO: 9)
5′-GCACTCAAGGCAAGCTTTATTGA-3′,
detection probe HIV-ZP2:
(SEQ ID NO: 10)
5′-GCTTAAGCAGTGGGTTCCCTAG-3′,
forward primer Z-POL-F2:
(SEQ ID NO: 11)
5′-CTCAGGTCACTCTTTGGCAACG-3′,
reverse primer Z-POL-R2:
(SEQ ID NO: 12)
5′-TCCTCCTATCATTTTTGGTTTCC-3′,
forward primer Z-POL-FO2:
(SEQ ID NO: 13)
5′-CAAATCCCTCTTTGGGACAGAC-3′,
and
detection probe Z-POL-P2:
(SEQ ID NO: 14)
5′-GTATCATCTGCTCCTGTATCT-3′;
internal standard forward primer HPGT31-F:
(SEQ ID NO: 25)
5′-CAAAGACCGTTGTGTCCAGAAG-3′,
internal standard reverse primer HPGT31-R:
(SEQ ID NO: 26)
5′-CGCATGTTTACACTTGGGTTTC-3′,
and
internal standard probe HPGT31-P:
(SEQ ID NO: 27)
5′-CAACGTCCTGTCCACCTTCCTCCTATG-3′.

Fluorescent groups of the detection probes were FAM, and fluorescent groups of the internal standard probe were HEX.

Notably, the sequences of the forward primers, reverse primers, and detection probes in Example 2 are from about 50% to about 80% identical to those of the corresponding forward primers, reverse primers, and detection probes in Example 1.

### Example 3. Still Another Set of Primers and Probes Used in the Present Disclosure

forward primer HIV-ZF3:
(SEQ ID NO: 15)
5′-TGGGAGCTCTCTGGCTAACTA-3′,
reverse primer HIV-ZR3:
(SEQ ID NO: 16)
5′-CTTGAAGCACTCAAGGCAAGCTTTA-3′,
detection probe HIV-ZP3:
(SEQ ID NO: 17)
5′-ATTGAGGCTTAAGCAGTGGGT-3′,
forward primer Z-POL-F3:
(SEQ ID NO: 18)
5′-GTCACTCTTTGGCAACGACCCCT-3′,
reverse primer Z-POL-R3:
(SEQ ID NO: 19)
5′-CCCCTATCATTTTTGGTTTCCATC-3′,
forward primer Z-POL-FO3:
(SEQ ID NO: 20)
5′-TCCCTCTTTGGGACAGACCAR-3′,
and
detection probe Z-POL-P3:
(SEQ ID NO: 21)
5′-AATACTGTATCATCTGCTCCTG-3′,
internal standard forward primer HPGT31-F:
(SEQ ID NO: 25)
5′-CAAAGACCGTTGTGTCCAGAAG-3′,
internal standard reverse primer HPGT31-R:
(SEQ ID NO: 26)
5′-CGCATGTTTACACTTGGGTTTC-3′,
and
internal standard probe HPGT31-P:
(SEQ ID NO: 27)
5′-CAACGTCCTGTCCACCTTCCTCCTATG-3′.

Fluorescent groups of the detection probes were FAM, and fluorescent groups of the internal standard probe were HEX.

Notably, the sequences of the forward primers, reverse primers, and detection probes in Example 3 are from about 50% to about 80% identical to those of the corresponding forward primers, reverse primers, and detection probes in Example 1.

### Example 4. Primers and Probes for the Internal Standard Used in the Present Disclosure

### (A) an Exogenous Non-Human Genomic Internal Standard:

forward primer HPGT31-F:
(SEQ ID NO: 25)
5′-CAAAGACCGTTGTGTCCAGAAG-3′,
reverse primer HPGT31-R:
(SEQ ID NO: 26)
5′-CGCATGTTTACACTTGGGTTTC-3′,
and
internal standard probe HPGT31-P:
(SEQ ID NO: 27)
5′-CAACGTCCTGTCCACCTTCCTCCTATG-3′.

### (B) An Exogenous Synthetic Internal Standard

forward primer NNJ-F:
(SEQ ID NO: 28)
5′-AGTACGCAACAAGTCGAACC-3′,
reverse primer NNJ-R:
(SEQ ID NO: 29)
5′-TGGAGGAGAAGGAAGACTGAGGAA-3′,
and
internal standard probe NNJ-P:
(SEQ ID NO: 30)
5′-CCCAGTGAGGACTACTCCGTTGGGGG-3′;
or

### (C) An Exogenous Endogenous Human Genomic Internal Standard

forward primer RNaseP-F:
(SEQ ID NO: 31)
5′-GCGGAGGGAAGCTCATCAG-3′,
reverse primer RNaseP-R:
(SEQ ID NO: 32)
5′-GGACATGGGAGTGGAGTGACA-3′,
and
internal standard probe RNaseP-P:
(SEQ ID NO: 33)
5′-CACGAGCTGAGTGCGTCCT-3′.

Fluorescent groups of the internal standard probe were HEX.

### Example 5. Method for Detecting HIV-1

Extraction of HIV-1 RNA nucleic acid: nucleic acid extraction or purification reagent produced by SANSURE BIOTECH INC.

The following step were performed according to the manufacturer's technical recommendations/protocols when possible.

5.1 About 20 μL proteinase K and about 500 μL lysis buffer were added to an appropriate number of 1.5 mL centrifuge tubes each.

5.2 After centrifugation at 6000 rpm for 5 min, about 800 μL of a sample (plasma) to be tested was added to the above centrifuge tube. The tube was capped and the solution was mixed by shaking for 10 sec.

5.3 After being shaken for 10 sec, the tube was allowed to stand for about 30 min.

5.4 After a short spin, the centrifuge tube was placed in the magnetic separator for about 5 min, and then the solution was gently pipetted out without touching the brown substance adsorbed on the tube wall.

5.5 About 500 μL of nucleic acid washing solution 1 and about 200 μl of nucleic acid washing solution 2 were added and mixed by shaking for 5 sec. After a short spin, the centrifuge tube was placed in the magnetic separator again.

5.6 After standing for about 3 min, the supernatant was divided into two layers. The pipette tip was inserted into the bottom of the centrifuge tube, and the liquid was gently pipetted out entirely from the bottom and discarded. After standing for 1 minute, the residual liquid at the bottom of the tube was pipetted out and discarded completely.

5.7 About 35 μL of elution solution was added to elute the magnetic beads from the wall to the bottom of the centrifuge tube. After mixed by pipetting for 3 to 4 times and allowed to stand at room temperature for 10 minutes, the centrifuge tube was placed in the separator again for 3 min. The eluted nucleic acid was placed in a new 1.5 mL sterile centrifuge tube.

About 30 μL of the eluted nucleic acid was pipetted to PCR reaction solution and subjected to PCR amplification for detection.

### Nucleic Acid PCR Amplification:

6.1 The PCR reaction tubes were put into sample wells of the amplifier with the negative control, positive control, quantitative reference products A-D and the unknown samples set in the corresponding order, and the name of the sample and the concentration of the quantitative reference product were set.

6.2 Fluorescence detection channel selection

6.2.1 ABI instrument and Hongshi SLAN instrument:

1) FAM channel (Reporter: FAM, Quencher: none) was selected to detect HIV-RNA; 2) HEX or VIC channel (Reporter: HEX/VIC, Quencher; none) was selected to detect HIV internal standard; and 3) Reference fluorescence (Reference Dye)): ROX was selected.

6.2.2 Roche fluorescent PCR instrument:

New Experiment option was selected, and Dual Color Hydrolysis Probe/UPL Probe was selected in the drop-down menu of Detection format on the setup panel. In the drop-down menu Customize: 1) FAM channel was selected to detect HIV-RNA; and 2) VIC/HEX/Yellow channel was selected to detect HIV internal standard. Reaction Volume was set to 60.

6.3 setup of the cycle parameter:

| | | | | Number |
|---|---|---|---|---|
| | Step | Temperature | Time | of cycles |
| 1 | Pre-denaturation and enzyme | 95° C. | 1 min | 1 |
| | activation | | | |
| 2 | Reverse transcription | 60° C. | 30 min | 1 |
| 3 | cDNA pre-denaturation | 95° C. | 1 min | 1 |
| 4 | Denaturation | 95° C. | 15 sec | 45 |
| | Annealing, extending and | 58° C. | 30 sec | |
| | fluorescence collecting | | | |
| 5 | Cooling instrument | 25° C. | 10 sec | 1 |

### Analysis of Results

After the reaction was completed, the results were automatically saved, and the curve for the human immunodeficiency virus and the curve for the corresponding human immunodeficiency virus internal standard were analyzed, respectively. According to the image after analysis, Start value, End value and Threshold value of Baseline were adjusted (Start value may be set at 3-15, End value may be set at 5-20, and the amplification curve for the negative control can be adjusted to be horizontal and straight or below the threshold line). After clicking Analyze button for analysis, the quantitative results were recorded in the Plate window.

### Example 6. Detection of Clinical Samples Using the Composition of the Present Application

Clinical samples were used in the experiment for comparison. A total of 500 plasma samples were tested, including 415 males and 85 females, with an age ranging from 10 to 80 years old. Various types of information were obtained by sequencing the samples, including 69 cases of B subtype (including B type and B′ type), 176 cases of BC subtype (including CRF07_BC and CRF08_BC type) and 140 cases of AE subtype (including CRF01_AE type). The composition in Example 1 and the control reagent were used for detection according to the method described in Example 5. The results are shown in Table 1.

TABLE 1
Distribution of results of clinical samples
tested with the present reagent

| | Number (case) of samples | Number (case) of samples |
|---|---|---|
| Test results | detected with the present | detected with the control |
| (IU/mL) | reagent | reagent |
| 1E7~1E8 | 8 | 7 |
| 1E6~1E7 | 38 | 38 |
| 1E5~1E6 | 107 | 109 |
| 1E4~1E5 | 110 | 108 |
| 1E3~1E4 | 63 | 63 |
| 250~1E3 | 46 | 44 |
| 20~250 | 83 | 85 |
| <20(negative) | 45 | 46 |

It can be seen from the results in Table 1 that the clinical samples can be accurately detected with the composition of the present application. Limited by the minimum detection limit, the control reagent has a low detection efficiency for samples with a viral load below 25 IU/mL, resulting in one more sample with a viral load below 25 IU/mL detected using the control reagent than that using the present reagent, that is, the number of the detected positive samples using the control reagent was cut down by one. However, using the composition of the present application, samples below 25 IU can be detected. therefore, no false negative occurred.

### Example 7. Sensitivity Tests of the Composition of the Present Application

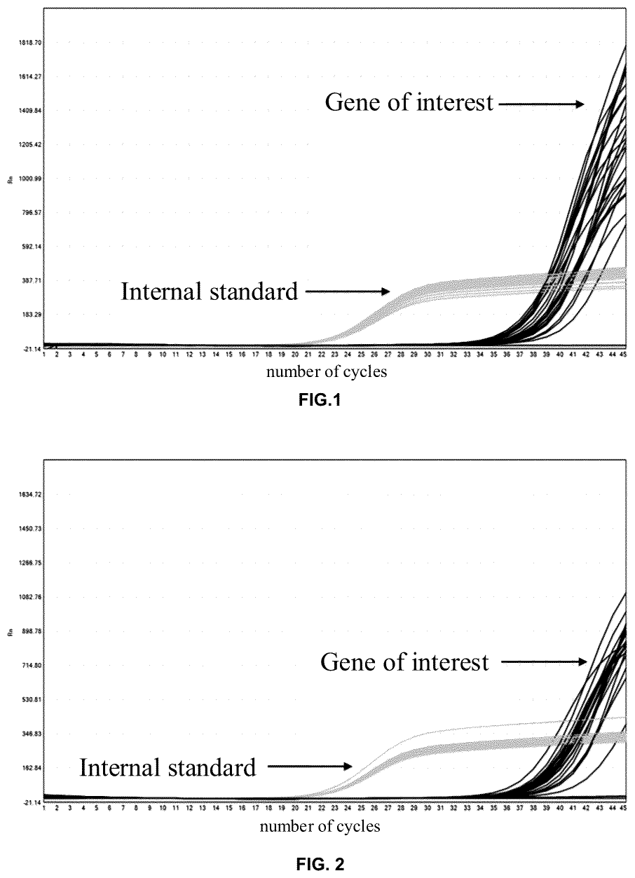

This test was performed by diluting the reference product of known concentration to 25 IU/mL and 22 IU/mL and using the composition in Example 1 according to the method described in Example 5. The results are shown in FIGS. 1-2. The experimental result shows that a sample of 22 IU/mL can be detected using the composition of the present application.

### Comparative Example 1. Detection of HIV-1 Using a Comparative Composition

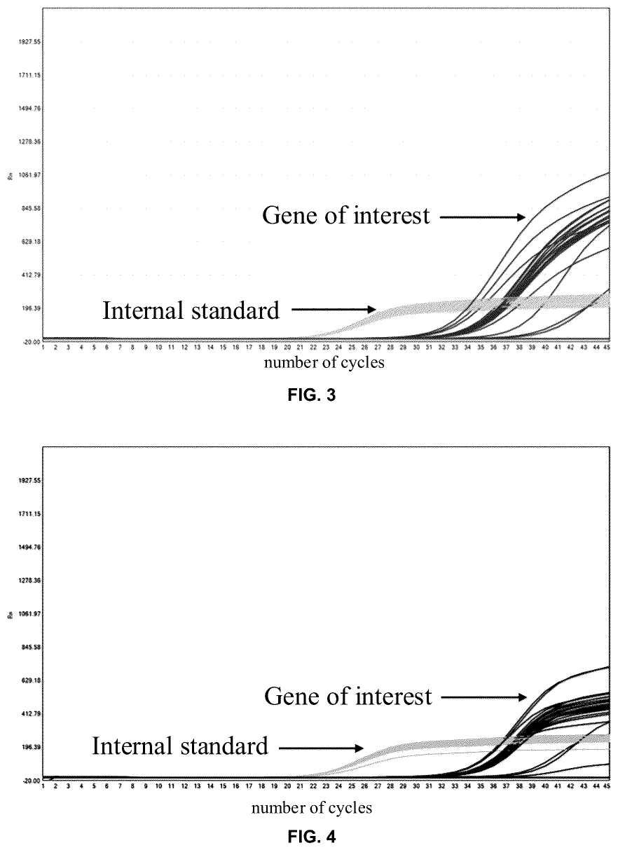

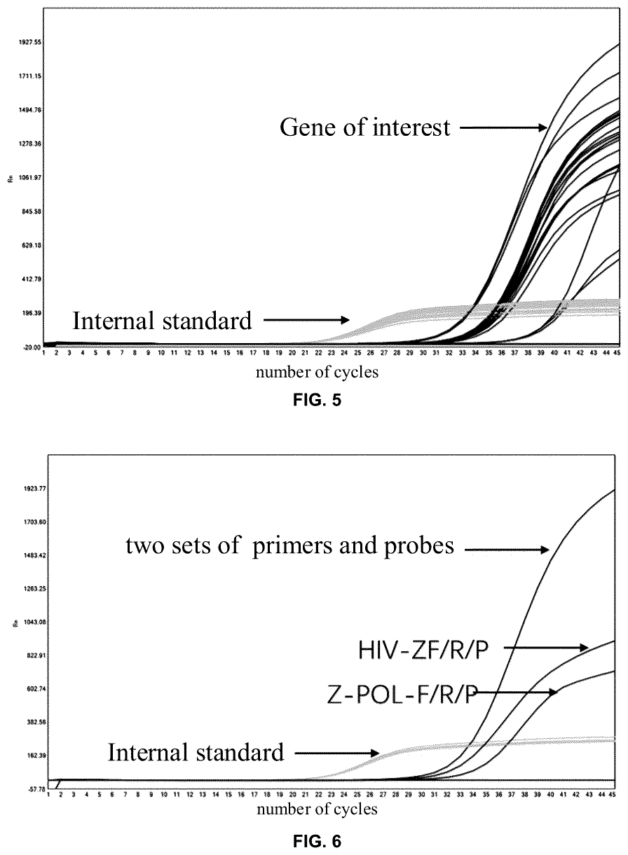

In this comparative experiment, clinical samples were used and 24 cases of plasma were tested. The composition in Example 1 and the control reagent were used for detection according to the method described in Example 5. The results are shown in FIGS. 3-6. Specifically, FIG. 3 shows the detection of clinical samples using a single set of primers and probe, HIV-ZF1/R1/P1. FIG. 4 shows the detection of clinical samples using a single set of primers and probe, Z-POL-F1/R1/P1. FIG. 5 shows an example of the detection of clinical samples using two sets of primers and probes, HIV-ZF1/R1/P1 and Z-POL-F1/R1/P1. FIG. 6 shows the detection of a single clinical sample using different combinations of primers and probes.

From the comparison results, it was found that the detection rate for amplification with single-target primer-probe were consistent with that for amplification with dual-target primer-probe combination. All 24 clinical samples could be detected. However, amplification curve with the single-target primer-probe has a lower fluorescence signal intensity than that with the dual-target primer-probe combination, and the detection curve with the single-target primer-probe has a delayed CT value compared with the dual-target primer-probe combination.

### Comparative Example 2. Comparative Detection of HIV-1 with or without Primer of SEQ ID NO: 6 Added

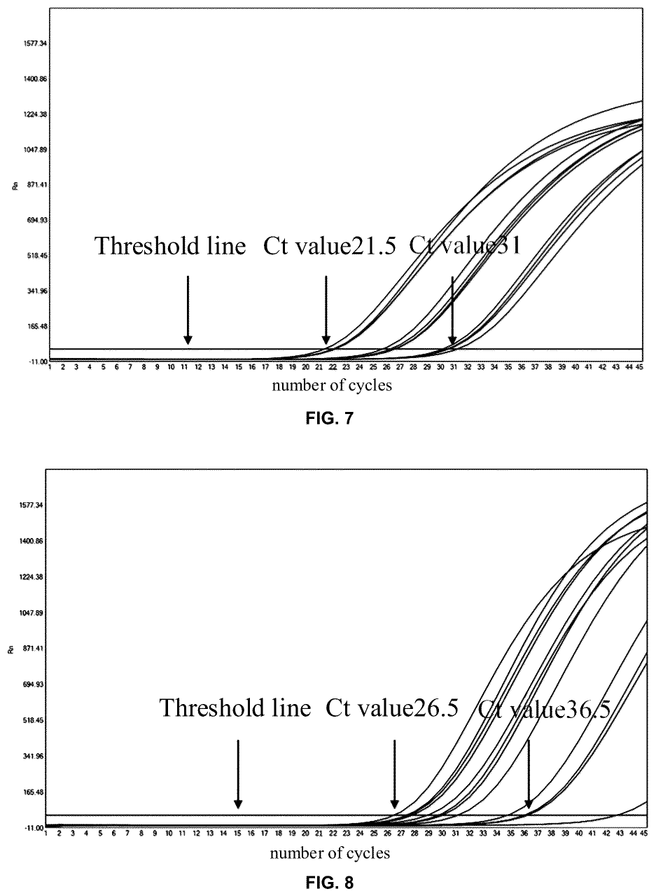

In this scheme, an HIV-1 group O reference sample at a determined concentration was diluted in equal proportion, and then detected using a dual detection system with or without the primer of SEQ ID NO: 6 added. The test results are shown in FIGS. 7-8. Specifically, FIG. 7 shows detection of a reference sample using two sets of primers and probes with the primer of SEQ ID NO: 6. FIG. 8 shows detection of a reference sample using two sets of primers and probes without the primer of SEQ ID NO: 6.

As a result of this scheme, with the primer of SEQ ID NO: 6, the HIV-1 group O reference sample was detected with a CT value between 21.5 and 26.5 and a detection rate of 100% (12/12); while without the primer of SEQ ID NO: 6 added, the HIV-1 group O reference sample at the same concentration was detected with a CT value between 26.5 and 36.5 and a lower detection rate of 91.7% (11/12). Moreover, addition of the primer of SEQ ID NO: 6 resulted in an average reduction of CT value by 5, a better curve, and a higher detection rate for samples at a low concentration, indicating that adding the primer of SEQ ID NO: 6 improves the sensitivity of the entire composition for detecting HIV, particularly HIV-1 group O, as compared to the detection without the primer of SEQ ID NO: 6.

### Comparative Example 3. Comparative Detection of HIV-1 Using Two Sets of Primers and Probes

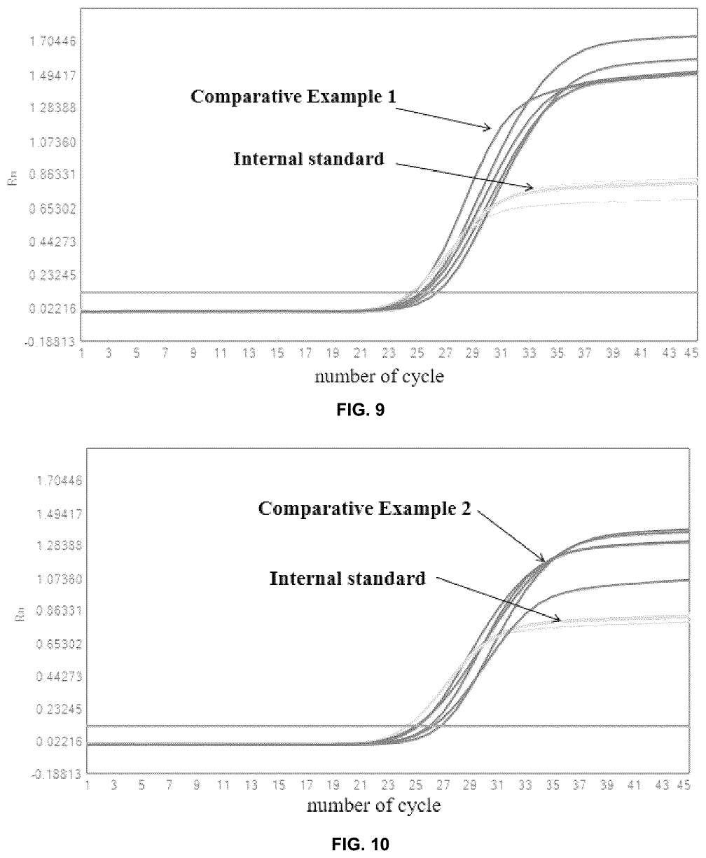

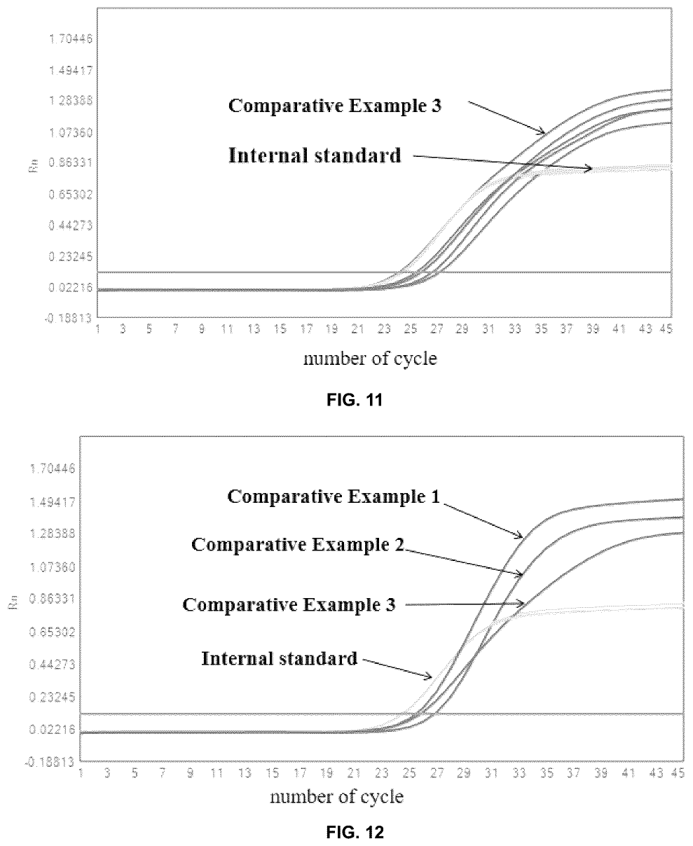

In this comparative experiment, clinical samples were used and 20 cases of plasma were tested. The compositions in Examples 1-3 and the control reagent were used for detection according to the method described in Example 5. The results are shown in FIGS. 9-12. Specifically, FIG. 9 shows an example of the detection of clinical samples using two sets of primers and probes, HIV-ZF1/R1/P1 and Z-POL-F1/R1/P1. FIG. 10 shows an example of the detection of clinical samples using two sets of primers and probes, HIV-ZF2/R2/P2 and Z-POL-F2/R2/P2. FIG. 11 shows an example of the detection of clinical samples using two sets of primers and probes, HIV-ZF3/R3/P3 and Z-POL-F3/R3/P3. FIG. 12 shows the detection of the same clinical sample using different two sets of primers and probes.

From the comparison results, it was found that the detection rate for amplification were consistent when using dual-target primer-probe combinations in Examples 1-3. Notably, the sequences of the forward primers, reverse primers, and detection probes in any one of the Examples 1-3 are from about 50% to about 80% identical to the corresponding forward primers, reverse primers, and detection probes in the other two of Examples 1-3.

All 20 clinical samples could be detected. However, amplification curve with the dual-target primer-probe combination in Example 1 has a higher fluorescence signal intensity than that with the dual-target primer-probe combinations in Examples 2-3, and the detection curve with the dual-target primer-probe combination in Example 1 has a higher CT value compared with the dual-target primer-probe combinations in Example 2-3.

### Comparative Example 4. Comparison of Experimental Results of Different Internal Standard Sequences

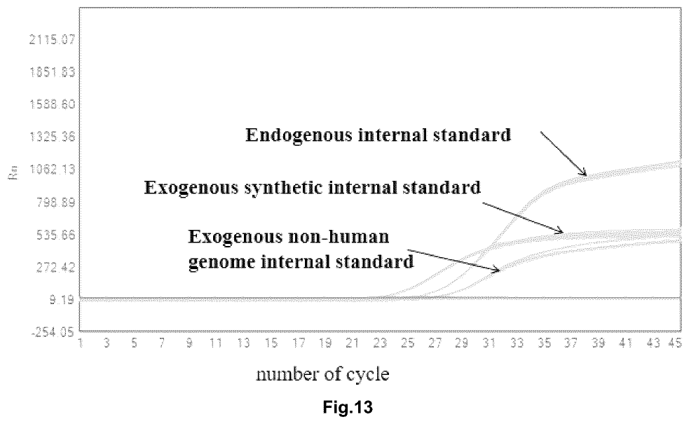

In this comparative experiment, exogenous non-human genomic internal standard, exogenous synthetic internal standard, and endogenous human genomic internal standard were tested 8 times, respectively. Detection was performed according to the method described in Example 5, wherein the concentrations of the three internal standards are different. Further, the exogenous non-human genomic internal standard primer/probe combination is SEQ ID NO: 25, SEQ ID NO: 26, and SEQ ID NO: 27, the primer probes for the exogenous synthetic internal standard are SEQ ID NO: 28, SEQ ID NO: 29, SEQ ID NO: 30, the primer probes for the endogenous human genome internal standard are SEQ ID NO: 31, SEQ ID NO: 32, and SEQ ID NO: 33, and the results are shown in FIG. 13.

From the comparison results, it was found that these three internal standards can be used as internal standard controls.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A composition for amplifying and detecting human immunodeficiency virus type 1 (HIV-1) nucleic acid sequence, comprising:
(a) a first set comprising a first forward primer, a first reverse primer, and a first single-stranded detection probe comprising a first label, wherein the first set amplifies and detects a first portion of the HIV-1 nucleic acid sequence, wherein the first portion is gag gene;
(b) a second set comprising a second forward primer, a second reverse primer, and a second single stranded detection probe comprising a second label, wherein the second set amplifies and detects a second portion of the HIV-1 nucleic acid sequence, wherein the second portion is pol gene; and
(c) a third forward primer, wherein the third forward primer amplifies a third portion of the HIV-1 nucleic acid sequence, wherein the third portion is a variant region of the HIV-1 nucleic acid sequence of pol gene.

2. The composition of claim 1, further comprising:
(d) an internal standard set comprising an internal standard forward primer, an internal standard revere primer, and a single-stranded internal standard detection probe, wherein the internal standard set amplifies and detects an internal standard if the internal standard is present.

3. The composition of claim 2, wherein the internal standard is an exogenous non-human genomic internal standard, an exogenous synthetic internal standard, or an endogenous human genomic internal standard.

4. The composition of claim 3, wherein the exogenous non-human genomic internal standard comprises a plant nucleic acid molecule, a fungi nucleic acid molecule, a bacteria nucleic acid molecule, an archaea nucleic acid molecule, or a virus nucleic acid molecule that is neither HIV-1 nor HIV type 2.

5. The composition of claim 4, wherein the exogenous non-human genomic internal standard is papillomavirus, Arabidopsis Scc3 homologue (AtSCC3), or histocompatibility complex class II beta chain paralogue (HLA-DRB3).

6. The composition of claim 3, wherein the endogenous human genomic internal standard is ribonuclease P (RnaseP), glyceraldehyde 3-phosphate dehydrogenase (GAPDH), β-actin, or 18S ribosomal ribonucleic acid (18S rRNA).

7. The composition of claim 1, wherein:
the first forward primer is at least 80% identical to a sequence selected from the group consisting of SEQ ID NOs: 1, 8 and 15;
the first reverse primer is at least 80% identical to a sequence selected from the group consisting of SEQ ID NOs: 2, 9 and 16;
the first detection probe is at least 80% identical to a sequence selected from the group consisting of SEQ ID NOs: 3, 10 or 17;
the second forward primer is at least 80% identical to a sequence selected from the group consisting of SEQ ID NOs: 4, 11 or 18;
the second reverse primer is at least 80% identical to a sequence selected from the group consisting of SEQ ID NOs: 5, 12 or 19; and
the second detection probe is at least 80% identical to a sequence selected from the group consisting of SEQ ID NOs: 7, 14 or 21.

8. The composition of claim 7, wherein:
the third forward primer is at least 80% identical to a sequence selected from the group consisting of SEQ ID NOs: 6, 13 or 20.

9. The composition of claim 1, wherein the first detection probe comprises a first detectable label, and wherein the second detection probe comprises a second detectable label.

10. The composition of claim 9, wherein the first detectable label is FAM, HEX, ROX, or CY5; and wherein the second detectable label is FAM, HEX, ROX, or CY5.

11. The composition of claim 10, wherein the first detection label is different from the second detection label.

12. A kit for amplifying and detecting human immunodeficiency virus type 1 (HIV-1) nucleic acid sequence, comprising:
(a) a first set comprising a first forward primer, a first reverse primer, and a first single-stranded detection probe comprising a first label, wherein the first set amplifies and detects a first portion of the HIV-1 nucleic acid sequence, wherein the first portion is gag gene;
(b) a second set comprising a second forward primer, a second reverse primer, and a second single stranded detection probe comprising a second label, wherein the second set amplifies and detects a second portion of the HIV-1 nucleic acid sequence, wherein the second portion is pol gene;
(c) a third forward primer, wherein the third forward primer amplifies a third portion of the HIV-1 nucleic acid sequence, wherein the third portion is a variant region of the HIV-1 nucleic acid sequence of pol gene;
(d) reagents for amplifying and detecting nucleic acid sequences; and
(e) instructions for use.

13. The kit of claim 12, further comprising:
(f) an internal standard set comprising an internal standard forward primer, an internal standard revere primer, and a single-stranded internal standard detection probe, wherein the internal standard set amplifies and detects an internal standard if the internal standard is present.

14. The kit of claim 12, wherein:
the first forward primer is at least 80% identical to a sequence selected from the group consisting of SEQ ID NOs: 1, 8 and 15;
the first reverse primer is at least 80% identical to a sequence selected from the group consisting of SEQ ID NOs: 2, 9 and 16;
the first detection probe is at least 80% identical to a sequence selected from the group consisting of SEQ ID NOs: 3, 10 or 17;
the second forward primer is at least 80% identical to a sequence selected from the group consisting of SEQ ID NOs: 4, 11 or 18;
the second reverse primer is at least 80% identical to a sequence selected from the group consisting of SEQ ID NOs: 5, 12 or 19;
the second detection probe is at least 80% identical to a sequence selected from the group consisting of SEQ ID NOs: 7, 14 or 21; and
the third forward primer is at least 80% identical to a sequence selected from the group consisting of SEQ ID NOs: 6, 13 or 20.

15. A method for detecting human immunodeficiency virus-1 (HIV-1) in a sample suspected of containing HV-1, the method comprising:
(i) contacting a sample with the composition of claim 1 and reagents for amplification and detection of nucleic acid sequences;
(ii) amplifying at least a portion of the gag gene present in the sample, thereby providing a first amplified sequence, and/or amplifying at least a portion of the pol gene present in the sample, thereby providing a second amplified sequence;
(iii) hybridizing the first single-stranded detection probe to the first amplified sequence, and/or hybridizing the second single-stranded detection probe to the second amplified sequence; and
(iv) detecting a first signal from hybridization of the first single-stranded detection probe to the first amplified sequence, and/or detecting a second signal from hybridization of the second single-stranded detection probe to the second amplified sequence.

16. The method of claim 15, further comprising:
in (ii), amplifying at least a portion of the variant region present in the sample, thereby providing a third amplified sequence;
in (iii), hybridizing the second single-stranded detection probe to the third amplified sequence; and
in (iv), detecting a third signal from hybridization of the second single-stranded detection probe to the third amplified sequence.

17. The method of claim 15, wherein a presence of the third amplified sequence is indicative of a presence of HIV-1 group O in the sample, and wherein absence of the third amplified sequence is indicative of an absence of HIV-1 group O in the sample.

18. The method of claim 15, further comprising:
in (i), contacting the sample with an internal standard set comprising an internal standard forward primer, an internal standard revere primer, and a single-stranded internal standard detection probe, wherein the internal standard set amplifies and detects an internal standard if the internal standard is present;
in (ii), amplifying at least a portion of the internal standard if present, thereby providing a fourth amplified sequence;
in (iii), hybridizing the single-stranded internal standard detection probe to the fourth amplified sequence; and
in (iv), detecting a fourth signal from hybridization of the single-stranded internal standard detection probe to the fourth amplified sequence.

19. The method of claim 15, wherein the sample comprises blood, serum, plasma, saliva, urine, vaginal fluid, or semen.

20. The method of claim 15, wherein a presence of either the first amplified sequence or the second amplified sequence is indicative of a presence of HIV-1 group M in the sample, and wherein absence of both the first amplified sequence and the second amplified sequence is indicative of an absence of HIV-1 group M in the sample.


SEQUENCE LISTING (33 sequences)

SEQ ID NO: 1 - DNA, 19 bp, synthetic construct
  source 1..19: mol_type = other DNA
cctgggagctctctggcta

SEQ ID NO: 2 - DNA, 22 bp, synthetic construct
  source 1..22: mol_type = other DNA
aagcactcaaggcaagctttat

SEQ ID NO: 3 - DNA, 20 bp, synthetic construct
  source 1..20: mol_type = other DNA
aggcttaagcagtgggttcc

SEQ ID NO: 4 - DNA, 21 bp, synthetic construct
  source 1..21: mol_type = other DNA
aaatcactctttggcarcgac

SEQ ID NO: 5 - DNA, 21 bp, synthetic construct
  source 1..21: mol_type = other DNA
ccccctatcatttttggtttc

SEQ ID NO: 6 - DNA, 20 bp, synthetic construct
  source 1..20: mol_type = other DNA
atccctctttgggacagacc

SEQ ID NO: 7 - DNA, 20 bp, synthetic construct
  source 1..20: mol_type = other DNA
actgtatcatctgcycctgt

SEQ ID NO: 8 - DNA, 20 bp, synthetic construct
  source 1..20: mol_type = other DNA
gagcctgggagctctctggc

SEQ ID NO: 9 - DNA, 23 bp, synthetic construct
  source 1..23: mol_type = other DNA
gcactcaaggcaagctttattga

SEQ ID NO: 10 - DNA, 22 bp, synthetic construct
  source 1..22: mol_type = other DNA
gcttaagcagtgggttccctag

SEQ ID NO: 11 - DNA, 22 bp, synthetic construct
  source 1..22: mol_type = other DNA
ctcaggtcactctttggcaacg

SEQ ID NO: 12 - DNA, 23 bp, synthetic construct
  source 1..23: mol_type = other DNA
tcctcctatcatttttggtttcc

SEQ ID NO: 13 - DNA, 22 bp, synthetic construct
  source 1..22: mol_type = other DNA
caaatccctctttgggacagac

SEQ ID NO: 14 - DNA, 21 bp, synthetic construct
  source 1..21: mol_type = other DNA
gtatcatctgctcctgtatct

SEQ ID NO: 15 - DNA, 21 bp, synthetic construct
  source 1..21: mol_type = other DNA
tgggagctctctggctaacta

SEQ ID NO: 16 - DNA, 25 bp, synthetic construct
  source 1..25: mol_type = other DNA
cttgaagcactcaaggcaagcttta

SEQ ID NO: 17 - DNA, 21 bp, synthetic construct
  source 1..21: mol_type = other DNA
attgaggcttaagcagtgggt

SEQ ID NO: 18 - DNA, 23 bp, synthetic construct
  source 1..23: mol_type = other DNA
gtcactctttggcaacgacccct

SEQ ID NO: 19 - DNA, 24 bp, synthetic construct
  source 1..24: mol_type = other DNA
cccctatcatttttggtttccatc

SEQ ID NO: 20 - DNA, 21 bp, synthetic construct
  source 1..21: mol_type = other DNA
tccctctttgggacagaccar

SEQ ID NO: 21 - DNA, 22 bp, synthetic construct
  source 1..22: mol_type = other DNA
aatactgtatcatctgctcctg

SEQ ID NO: 22 - DNA, 475 bp, papillomavirus
  source 1..475: mol_type = genomic DNA
ttgtaagtgtgaagccagaattgagctagtagtagaaagctcagcagacgaccttcgagcattccagcagctgtttctgaacaccctgtcctttgtgtgtccgtggtgaagagtttattaatgaagatattgtaaatttgcccgagaatgtcgattggagaaaaaaaggagctgtcactcccgtaaaacatcagggttcatgcggtagttgttgggcattctcggccgttgcaactgtagagggaataaataagattagaactggaaaattagtagaattatcagagcaagaacttgttgactgtgaaagacgtagccatggggcaaagaccgttgtgtccagaagaaaaacaaagacatttggataaaaagaaacgattccacaacataggaggaaggtggacaggacgttgcatagcatgttggagaagacctcgtactgaaacccaagtgtaaacatgcgatattttatact

SEQ ID NO: 23 - DNA, 738 bp, synthetic construct
  source 1..738: mol_type = other DNA
ttcatactcatggcgaagtacgcaacaagtcgaaccctcaatcccagtgaggactattgggggttacttcgcaaggacgccgcatgtgcagttcctcagtcttccttctcctccatatgaagatttatcccatacggtgctagcgaagcaagtcttccgagttcacatctcgaacgaaggtcactcacagttccgaaggtcactacggtttccgagtcttattgtttcttccacgtatgacagataccctcaaccctcaggcccacggggggttgctacgcccagggctactccgttggccctgaagggttacttcgttagggctacttggaggagttgaccaacccgaacatggtgccttgatcttcagctttccaataagaagatcattgatgcagcacactgagctaaattcttaaactatctttaccggtgtgctgcctcgcagagattctcaacctagtccgcgcatactctttccgtttctgtcttacagtctttcttgttcttcttttccctatagttagaagatgagcccctggggtgccctgcccttcttttacggctgatggacattcaatagatttatctgctgttgcctgatctacgaccccctcaatctgtcgaagcaaatgctgcacagagggagaagttgcctgaccgggctattcccttgttatctgttgtccaactggttgaatagtaatctgtgctggatccctctgaagctggtcattc

SEQ ID NO: 24 - DNA, 341 bp, Homo sapiens
  source 1..341: mol_type = genomic DNA
atagggcggagggaagctcatcagtggggccacgagctgagtgcgtcctgtcactccactcccatgtcccttgggaaggtctgagactagggccagaggcggccctaacagggctctccctgagcttcggggaggtgagttcccagagaacggggctccgcgcgaggtcagactgggcaggagatgccgtggaccccgcccttcggggaggggcccggcggatgcctcctttgccggagcttggaacagactcacggccagcgaagtgagttcaatggctgaggtgaggtaccccgcaggggacctcataacccaattcagactactctcctccgcccatt

SEQ ID NO: 25 - DNA, 22 bp, synthetic construct
  source 1..22: mol_type = other DNA
caaagaccgttgtgtccagaag

SEQ ID NO: 26 - DNA, 22 bp, synthetic construct
  source 1..22: mol_type = other DNA
cgcatgtttacacttgggtttc

SEQ ID NO: 27 - DNA, 27 bp, synthetic construct
  source 1..27: mol_type = other DNA
caacgtcctgtccaccttcctcctatg

SEQ ID NO: 28 - DNA, 20 bp, synthetic construct
  source 1..20: mol_type = other DNA
agtacgcaacaagtcgaacc

SEQ ID NO: 29 - DNA, 24 bp, synthetic construct
  source 1..24: mol_type = other DNA
tggaggagaaggaagactgaggaa

SEQ ID NO: 30 - DNA, 26 bp, synthetic construct
  source 1..26: mol_type = other DNA
cccagtgaggactactccgttggggg

SEQ ID NO: 31 - DNA, 19 bp, synthetic construct
  source 1..19: mol_type = other DNA
gcggagggaagctcatcag

SEQ ID NO: 32 - DNA, 21 bp, synthetic construct
  source 1..21: mol_type = other DNA
ggacatgggagtggagtgaca

SEQ ID NO: 33 - DNA, 19 bp, synthetic construct
  source 1..19: mol_type = other DNA
cacgagctgagtgcgtcct